(19) United States
(12) Patent Application Publication
(10) Pub. No.: US 2022/0129427 A1
(43) Pub. Date: Apr. 28, 2022

(54) A KIND OF MONITORING METHOD FOR DRINKING BEHAVIOR OF LABORATORY MICE, AND ITS SYSTEM AND DEVICE

(71) Applicant: Nanjing Medical University, Nanjing City (CN)

(72) Inventors: Qigang Zhou, Nanjing City (CN); Chunyu Yin, Nanjing City (CN); Dongya Zhu, Nanjing City (CN); Feng Han, Nanjing City (CN); Liandi Li, Nanjing City (CN); Peijin Xu, Nanjing City (CN)

(21) Appl. No.: 17/253,199

(22) PCT Filed: Jan. 3, 2020
(86) PCT No.: PCT/CN2019/125005

§ 371 Date: Dec. 17, 2020

(30) Foreign Application Priority Data
Jul. 15, 2019 (CN) 2019106366090

(51) Int. Cl. G06F 16/215; G06F 16/22

(52) U.S. Cl. CPC G06F 16/215; G06F 16/2228

(57) ABSTRACT

The embodiment of the present invention discloses a kind of monitoring method for drinking behavior of laboratory mice, and its system and device, and thus obtains raw data of the drinking behavior of mice, and then stores the raw data; as well as conducts data filtering on the raw data to filter out false data of jitter from the raw data, wherein, the false data of jitter consists of the data triggered by measurement signal transient and jitter caused by accidentally touch of the mice body, and their exploratory action; and then completes index statistics and analysis of the raw data filtered out false data of jitter, and thus forms indicator data, as well as stores the indicator data; after that, the indicator data is displayed in classification of real-time data, historical data and report data. The present invention utilizes the serial filtering mechanism of data filtering and matching, and thus reduces the amount of invalid trigger data, and there is no accumulated performance loss of data processing, as well as has efficient, accurate and real processing capabilities to process experimental batch data, and can provide reliable and objective statistical indicators to laboratory personnel, as well as may detect multiple laboratory mice at the same time, and thus, greatly improves the efficiency of the experiment and the accuracy of the experimental results.

### FIELD OF THE INVENTION

The embodiments of the present invention relate to the technical field of data processing, particularly to a kind of monitoring method for drinking behavior of laboratory mice, and its system and device.

### BACKGROUND OF THE INVENTION

As one of the most popular laboratory animals, mice plays an irreplaceable role in the process of understanding of life phenomena and mechanisms of life by people, also enjoys an unparalleled position in the field of basic medical research. At present, the monitoring of the drinking behavior of laboratory mice is of great importance. For the purpose of obtaining accurate experimental result, it is necessary to collect and process the drinking behavior data of mice accurately, and thus ensure the accuracy and validity of the experimental result.

At the present stage, the monitoring of water intake of mice usually adopts manual timed monitoring and recording, which wastes a lot of time and effort of staff. There are also schemes which use light activated switches and/or licking recording system to collect data on time. These systems can report weight changes at specified time intervals, and cannot accurately monitor the drinking time and frequency of the mice. The existing technology can only report the intake at a fixed point-in-time, while cannot take the behavior factor of ingestion of animals into account. Furthermore, the traditional technical schemes still have more false data, and thus the accuracy of monitoring is low.

### SUMMARY OF THE INVENTION

For this purpose, the embodiments of the present invention provide a kind of monitoring method for drinking behavior of laboratory mice, and its system and device, which solves the problem that the processing procedure of monitoring data related to the number of times and the duration of drinking of mice is interfered by false data, and thus saves the effort of manual monitoring and completes the monitoring of drinking data of the laboratory mice accurately and efficiently.

In order to achieve the above purpose, the embodiments of the present invention provide following technical scheme: a monitoring method for drinking behavior of laboratory mice, which consists of following steps:

Data collection: to obtain the raw data of drinking of mice, and store the said raw data;

Data filtering: to conduct data filtering on the said raw data to filter out false data of jitter from the said raw data, wherein, the said false data of jitter consists of the data triggered by measurement signal transient and jitter caused by accidentally touch of the mice body, and their exploratory action;

Data statistics: to complete index statistics and analysis of the raw data filtered out the said false data of jitter, and thus form indicator data, as well as store the said indicator data;

Data presentation: the said indicator data is displayed in classification of real-time data, historical data and report data.

As a preferred scheme of the monitoring method for drinking behavior of laboratory mice, during the said data collection process, the communication parameter is set and the communication port is identified according to the selected operation object, and then receives the data collection instructions; after that, determines the validity of the instruction according to the current status of the said operation object and the mutual exclusion status among the objects, and thus, integrates effective instructions into a data packet of instructions for execution.

As a preferred scheme of the monitoring method for drinking behavior of laboratory mice, the status data and sampling data are fed back during the executive process of the said data packet of instructions, and then, inputs the said status data and sampling data in the data buffer pool; and detects the integrity and correctness of the said data packet of instructions, as well as analyzes the valid data points of the said instruction data for utilization.

As a preferred scheme of the monitoring method for drinking behavior of laboratory mice, during the said data filtering process, a non-false judgment method is used to filter the data triggered by measurement signal transient and jitter caused by accidentally touch of the mice body, and their exploratory action, and the statistical model of real drinking behavior of mice is used to match the real drinking data of mice.

As a preferred scheme of the monitoring method for drinking behavior of laboratory mice, during the said data presentation process, the data presentation methods include in the forms of animation, numerical value, list, curve graph, pie chart, image, and document.

The embodiments of the present invention also provide a kind of monitoring system for drinking behavior of laboratory mice, consists of:

A data collection module, which is used to obtain the raw data of drinking of mice, and store the said raw data;

A data filtering module, which is used to conduct data filtering on the said raw data to filter out false data of jitter from the said raw data, wherein, the said false data of jitter consists of the data triggered by measurement signal transient and jitter caused by accidentally touch of the mice body, and their exploratory action;

A data statistics module, which is used to complete index statistics and analysis of the raw data filtered out the said false data of jitter, and thus form indicator data, as well as store the said indicator data;

A data presentation module, which is used to display the said indicator data in classification of real-time data, historical data, and report data.

As a preferred scheme of the monitoring system for drinking behavior of laboratory mice, the said data collection module is equipped with a data transceiver unit and a validity judgment unit, and the said data transceiver unit is used to set communication parameter and identify communication port according to the selected operation object, and then receive data collection instructions; after that, the said validity judgment unit is used to determine the validity of the instruction according to the current status of the said operation object and the mutual exclusion status among the objects, and then, integrate effective instructions into a data packet of instructions for execution.

As a preferred scheme of the monitoring system for drinking behavior of laboratory mice, the said data collection module is further equipped with a data buffer unit, a data detection unit and a data analysis unit, wherein, the said data buffer unit is used to feed back the status data and sampling data during the executive process of the said data packet of instructions, and then, inputs the said status data and sampling data in the data buffer pool; and the said data detection unit is used to detect the integrity and correctness of the said data packet of instructions, as well as the said data analysis unit is used to analyze the valid data points of the said instruction data for utilization.

As a preferred scheme of the monitoring system for drinking behavior of laboratory mice, the said data filtering module consists of a false data filtering unit and a data matching unit, wherein, the said false data filtering unit is used to filter the data triggered by measurement signal transient and jitter caused by accidentally touch of the mice body, and their exploratory action through non-false judgment method; and the said data matching unit is used to match the real drinking data of mice by using the statistical model of real drinking behavior of mice.

As a preferred scheme of the monitoring system for drinking behavior of laboratory mice, the said data presentation methods include in the forms of animation, numerical value, list, curve graph, pie chart, image, and document.

The embodiments of the present invention also provide a kind of detection device for drinking behavior of laboratory mice, which consists of:

A box body, and the said box body is divided into at least two chambers by at least one insert plate, and the said chambers are used to accommodate animals and allow the said animals to move around, furthermore, each of the said chambers is equipped with at least two fixing holes on the side walls, and a fixing frame is arranged above each said fixing hole;

Each of the said chambers is equipped with steps, and the said steps are arranged on one side close to the said fixing hole;

Four feeding bottles at least, wherein, one end of each of the said feeding bottle is connected to one of the said fixing frame, and the other end is arranged in the said chamber through the said fixing hole under the said fixing frame;

Four sensors at least, with function of data collection, and each of the said feeding bottles is equipped with the said sensor, and the said sensor is arranged in the said chamber close to the water nozzle of the said feeding bottle, which is used to sense the fluctuation of the liquid level at the said water nozzle, and record the number of times and the duration of the fluctuation of the water level;

A box cover, and the said box cover is arranged on the said box body and is flexibly connected to the box body.

1. As a preferred scheme of the detection device for drinking behavior of laboratory mice, the said detection device for behavior of animal further consists of four blockers at least, and each of the said feeding bottle is equipped with one said blocker, and the said blocker is arranged on the said steps and close to the said feeding bottle.

As a preferred scheme of the detection device for drinking behavior of laboratory mice, the said feeding bottle consists of a feeding tube and a liquid storage tube, and the end of the said feeding tube is connected to the end of the liquid storage tube, and the said feeding tube passes through the said fixing hole and then is arranged in the said chamber, and the said liquid storage tube is clamped with the said fixing frame.

As a preferred scheme of the detection device for drinking behavior of laboratory mice, the said water nozzle is arranged at one end of the feeding tube far away from the said liquid storage tube.

As a preferred scheme of the detection device for drinking behavior of laboratory mice, the said feeding tube and the said liquid storage tube are vertically connected to each other.

As a preferred scheme of the detection device for drinking behavior of laboratory mice, the said box body and the said insert plate are made of non-transparent material.

As a preferred scheme of the detection device for drinking behavior of laboratory mice, the said box cover is arranged a number of vents.

The embodiments of the present invention have the following advantages: obtains raw data of drinking of mice, and then stores the raw data; as well as conducts data filtering on the raw data to filter out false data of jitter from the raw data, wherein, the false data of jitter consists of the data triggered by measurement signal transient and jitter caused by accidentally touch of the mice body, and their exploratory action; and then completes index statistics and analysis of the raw data filtered out false data of jitter, and thus forms indicator data, as well as stores the indicator data; after that, the indicator data is displayed in classification of real-time data, historical data and report data. The present invention utilizes the serial filtering mechanism of data filtering and matching, and thus reduces the amount of invalid trigger data, and there is no accumulated performance loss of data processing, as well as has efficient, accurate and real processing capabilities to process experimental batch data, and can provide reliable and objective statistical indicators to laboratory personnel, as well as may detect multiple laboratory mice at the same time, and thus, greatly improves the efficiency of the experiment and the accuracy of the experimental results.

### BRIEF DESCRIPTION OF THE DRAWINGS

For the purpose of more clearly illustrating the embodiments of the present invention or the technical schemes in the prior art, the text below will briefly introduce the drawings that need to be used in the description of the embodiments or the prior art. It is obviously that the drawings described below are only exemplary. For those of ordinary skill in the art, other implementation drawings can be derived from the provided drawings without doing creative work.

FIG. 1 is a schematic diagram of a kind of monitoring method for drinking behavior of laboratory mice provided in the embodiment of the present invention;

FIG. 2 is a schematic diagram of data collection of a kind of monitoring method for drinking behavior of laboratory mice provided in the embodiment of the present invention;

FIG. 3 is a schematic diagram of data filtering of a kind of monitoring method for drinking behavior of laboratory mice provided in the embodiment of the present invention;

FIG. 4 is a schematic diagram of a kind of detection system for drinking behavior of laboratory mice provided in the present invention;

FIG. 5 is an interface diagram of the monitoring software for drinking behavior of laboratory mice developed based on the technical scheme of the present invention.

FIG. 6 is a schematic diagram of the detection device for drinking behavior of laboratory mice provided by the embodiment 1 of the present invention;

FIG. 7 is a schematic diagram of the first view of the chamber provided in the embodiment 1 of the present invention;

FIG. 8 is a schematic diagram of the second view of the chamber provided in the embodiment 1 of the present invention;

FIG. 9 is a schematic diagram of the feeding bottle provided in the embodiment 1 of the present invention;

FIG. 10 is the result of water preference experiment conducted in the experimental example 1 of the present invention;

FIG. 11 is the result of water preference experiment conducted in the experimental example 2 of the present invention;

FIG. 12 is the result of water preference experiment conducted in the experimental example 3 of the present invention;

FIG. 13 is the result of water preference experiment conducted in the experimental example 4 of the present invention.

### DETAILED DESCRIPTION OF THE INVENTION

The text below explained the mode of execution of the present invention through specific embodiments. For those skilled in the art, the other advantages and effects of the present invention can be easily understood based on the content disclosed in this specification. It is obviously that the described embodiments are part of the embodiments of the present invention, rather than being all examples. Based on the embodiments of the present invention, all other embodiments obtained by those of ordinary skill in the art without doing creative work shall fall within the protection scope of the present invention.

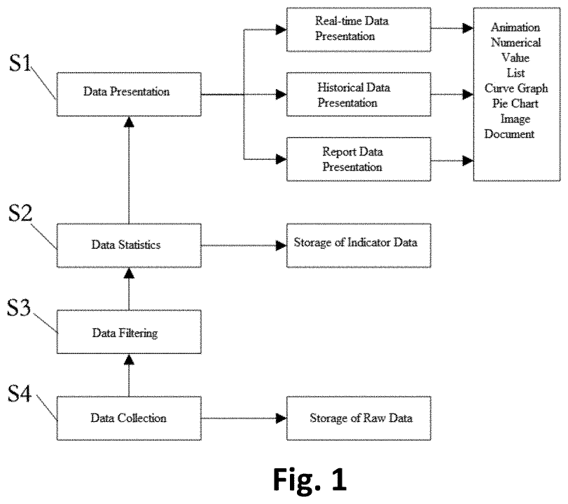

Referring to FIG. 1, a monitoring method for drinking behavior of laboratory mice is provided, which consists of following steps:

S1: Data collection: to obtain the raw data of drinking of mice, and store the said raw data;

S2: Data filtering: to conduct data filtering on the said raw data to filter out false data of jitter from the said raw data, wherein, the said false data of jitter consists of the data triggered by measurement signal transient and jitter caused by accidentally touch of the mice body, and their exploratory action;

S3: Data statistics: to complete index statistics and analysis of the raw data filtered out the said false data of jitter, and thus form indicator data, as well as store the said indicator data;

S4: Data presentation: the said indicator data is displayed in classification of real-time data, historical data, and report data.

Specifically, the collected raw data is firstly saved as a basis for backtracking; and the raw data needs to be processed by data filtering to filter out false data of jitter, and provide the upper layer with data of real drinking action; and then perform indicator analysis and statistics according to experimental requirements, and save the calculated index data in the database; as well as present the experimental results to the laboratory personnel in various forms shown in the figure, and thus provide convenience for the laboratory personnel to use.

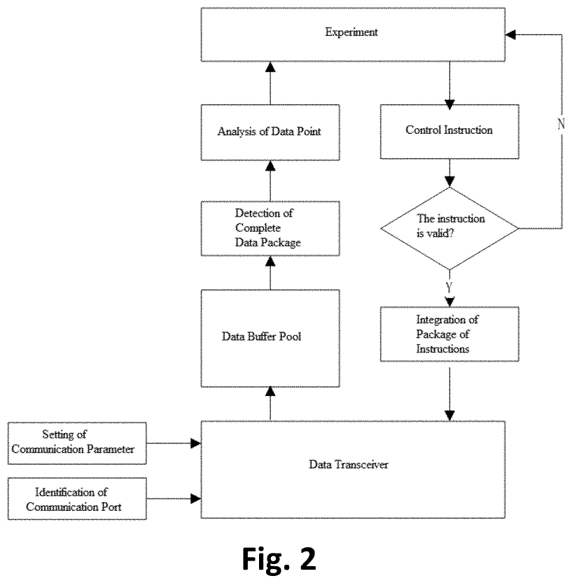

Referring to FIG. 2, in an embodiment of the monitoring method for drinking behavior of laboratory mice, during the said data collection process, the communication parameter is set and the communication port is identified according to the selected operation object, and then receives the data collection instructions; after that, determines the validity of the instruction according to the current status of the said operation object and the mutual exclusion status among the objects, and thus, integrates effective instructions into a data packet of instructions for execution. Furthermore, the status data and sampling data are fed back during the executive process of the said data packet of instructions, and then, inputs the said status data and sampling data in the data buffer pool; and detects the integrity and correctness of the said data packet of instructions, as well as analyzes the valid data points of the said instruction data for utilization.

Specifically, the laboratory personnel select the operation object and control the start, pause, or stop status. During the data collection process, the received data collection determines the validity of the instruction according to the current status of the operation object and the mutual exclusion status among the objects, and then, integrates effective instructions into a data packet of instructions, as well as sends the data packet to the equipment for execution. The feedback status data and sampling data are dropped into the data buffer pool, and then, detects the complete and correct data packets through the data packet detection technology, as well as analyzes and calculates valid data points, and thus, provides to the upper layer for use.

In an embodiment of the monitoring method for drinking behavior of laboratory mice, during the said data filtering process, a non-false judgment method is used to filter the data triggered by measurement signal transient and jitter caused by accidentally touch of the mice body, and their exploratory action, and the statistical model of real drinking behavior of mice is used to match the real drinking data of mice. Furthermore, during the said data presentation process, the data presentation methods include in the forms of animation, numerical value, list, curve graph, pie chart, image, and document.

Specifically, the serial mechanism of filtering non-false and matching the real is applied to ensure the real drinking data is obtained. In practice, due to the influence of the device space and the probe to the radiation, the flexibility of the mice body, the movement, the posture, and the action etc., the phenomenon of tail sweeping, false triggering of the body, and their exploratory action, etc, which may cause the trigger action by measurement signal transient and jitter. However, the filtered signal cannot be guaranteed to be true, which shall be matched through the real drinking statistical model to obtain the real and valid data to the maximum extent.

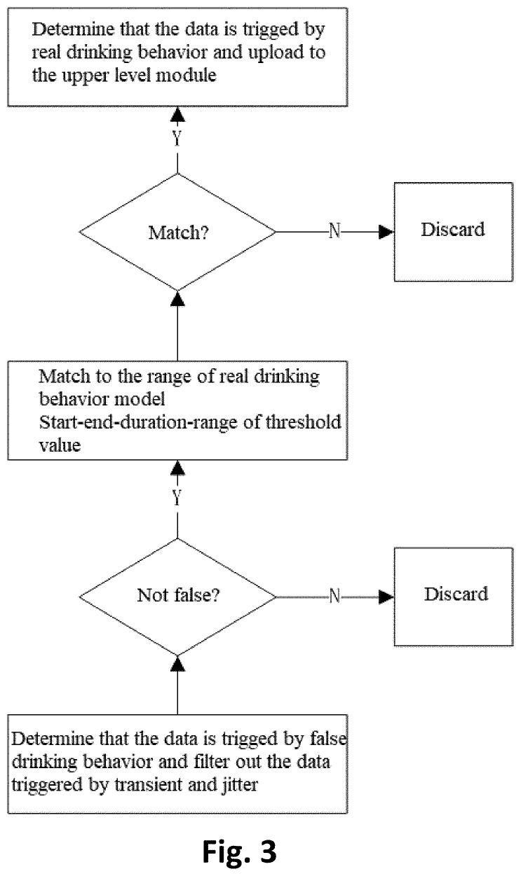

Referring to FIG. 3, specifically, in the process of the mice experiment, the observation time is generally long, and the number of mice is large, as well as the amount of data is large, which is necessary to rely entirely on the device to automatically obtain valid data and calculate drinking indicators. It is required to ensure that every trigger of drinking action will not be missed, delayed, or falsely alarmed. This technical scheme adopts a serial mechanism of filtering non-false and matching the real to ensure that the real drinking data is obtained, which can better meet the needs of the real situation. Wherein, the non-false judgment processing is an exclusion method which can filter out unreal data at the shallow level, but cannot guarantee that the data passed is true. In addition, the matching processing is a kind of screening method which can filter out the real data within expectations, but cannot guarantee that the unexpected data failed. This technical scheme combines the non-false processing and the matching processing in series, and the dual mechanism can obtain the real and valid data to the maximum extent.

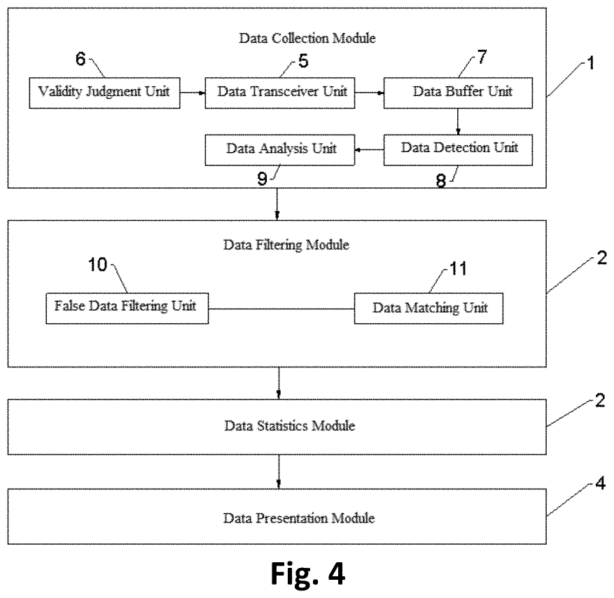

Referring to FIG. 4, an embodiment of the present invention also provides a monitoring system for drinking behavior of laboratory mice, consists of:

The data collection module 1, which is used to obtain the raw data of drinking of mice, and store the said raw data;

The data filtering module 2, which is used to conduct data filtering on the said raw data to filter out false data of jitter from the said raw data, wherein, the said false data of jitter consists of the data triggered by measurement signal transient and jitter caused by accidentally touch of the mice body, and their exploratory action;

The data statistics module 3, which is used to complete index statistics and analysis of the raw data filtered out the said false data of jitter, and thus form indicator data, as well as store the said indicator data;

The data presentation module 4, which is used to display the said indicator data in classification of real-time data, historical data, and report data.

In an embodiment of the monitoring system for drinking behavior of laboratory mice, the said data collection module 1 is equipped with a data transceiver unit 5 and a validity judgment unit 6, and the said data transceiver unit 5 is used to set communication parameter and identify communication port according to the selected operation object, and then receive data collection instructions; after that, the said validity judgment unit 6 is used to determine the validity of the instruction according to the current status of the said operation object and the mutual exclusion status among the objects, and then, integrate effective instructions into a data packet of instructions for execution.

In an embodiment of the monitoring system for drinking behavior of laboratory mice, the said data collection module 1 is further equipped with a data buffer unit 7, a data detection unit 8 and a data analysis unit 9, wherein, the said data buffer unit 7 is used to feed back the status data and sampling data during the executive process of the said data packet of instructions, and then, input the said status data and sampling data in the data buffer pool; and the said data detection unit 8 is used to detect the integrity and correctness of the said data packet of instructions, as well as the said data analysis unit 9 is used to analyze the valid data points of the said instruction data for utilization.

In an embodiment of the experimental mice drinking water monitoring system, the said data filtering module 2 consists of a false data filtering unit 10 and a data matching unit 11, wherein, the said false data filtering unit 10 is used to filter the data triggered by measurement signal transient and jitter caused by accidentally touch of the mice body, and their exploratory action through non-false judgment method; and the said data matching unit 11 is used to match the real drinking data of mice by using the statistical model of real drinking behavior of mice. During the said data presentation process, the data presentation methods include in the forms of animation, numerical value, list, curve graph, pie chart, image, and document.

Specifically, a set of equipment was designed and a set of software was developed based on the method and system technical scheme of the present invention to detect the drinking behavior of mice. The box body of the device is divided into at least 5 chambers by at least one insert plate, which can test at least 5 mice at the same time. In addition, quite a lot of sensors are arranged on the liquid level of the water bottle to monitor the drinking behavior of mice. The sensor is arranged in the chamber, and close to the water nozzle of the feeding bottle, which is used to sense the fluctuation of the liquid level at the water nozzle. Furthermore, the time and frequency of drinking by mice is accurately recorded, and thus exactly learn the behavior of mice. When the mice drink water, they need to climb the steps, and there are at least four sensors are required to avoid drinking action caused by voluntary activities of mice. Each feeding bottles is equipped with a sensor, and the sensor is arranged in the chamber and close to the water nozzle of the feeding bottle, which is used to sense the fluctuation of the liquid level at the said water nozzle, and record the number of times and the duration of the fluctuation of the water level. In order to aim at the fact that the device technology cannot solve the problem of calculating the number of times and the length of time of the drinking of mice. Referring to FIG. 5, based on the monitoring technical scheme for detecting the drinking of mice of the present invention, the effort of manual monitoring can be saved and it uses compute software to complete the calculating of the number of times and the length of time of the drinking of mice easily.

Specifically, in practice, the sensor uses E3S-GS30E4, and the power supply voltage is: DC12˜24V; the consumption current ≤40 mA; the response time ≤2 ms; the output interface: and the voltage output are NPN, PNP; the output current ≤100 mA. In addition, the sensitivity control is realized by adjusting the sensitivity potentiometer, and thus can change the resolution of the detected object.

Based on comparison test which requires to detect multiple mice drinking water at the same in one-time, the system networking mode is adopted, wherein, multiple detection units are combined into a detection system, and the networking units need to be arranged serial numbers. Thus, the dial switch is used for selection herein. The dial switch is used to operate the switch controlling addresses, which uses the principle of 0/1 binary coding. There are two pins on the top and bottom of the reverse side corresponding to each key. When the switch is pushed to the ON side, the two pins below are connected; otherwise, they are disconnected. Due to this experiment adopts the networking mode to sample the drinking condition of multiple mice, the 485-bus mode is used to communicate with the data organization board. Considering that the real-time data of multiple mice drinking water at the same time is large and complicated, the data collected by the sensor is sent to the host computer through the industrial serial port for organization and displayed visually, and thus makes the experimental data and results obvious at a glance.

The embodiment of the present invention obtains raw data of the drinking behavior of mice, and then stores the raw data; as well as conducts data filtering on the raw data to filter out false data of jitter from the raw data, wherein, the false data of jitter consists of the data triggered by measurement signal transient and jitter caused by accidentally touch of the mice body, and their exploratory action; and then completes index statistics and analysis of the raw data filtered out false data of jitter, and thus forms indicator data, as well as stores the indicator data; after that, the indicator data is displayed in classification of real-time data, historical data and report data. The present invention utilizes the serial filtering mechanism of data filtering and matching, and thus reduces the amount of invalid trigger data, and there is no accumulated performance loss of data processing, as well as has efficient, accurate and real processing capabilities to process experimental batch data, and can provide reliable and objective statistical indicators to laboratory personnel, as well as may detect multiple laboratory mice at the same time, and thus, greatly improves the efficiency of the experiment and the accuracy of the experimental results.

Embodiment 1

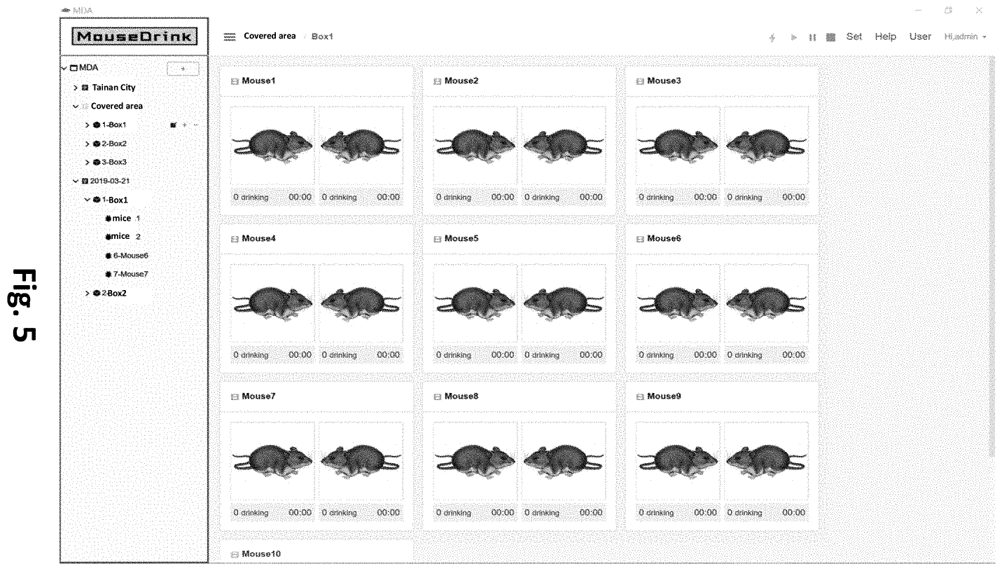

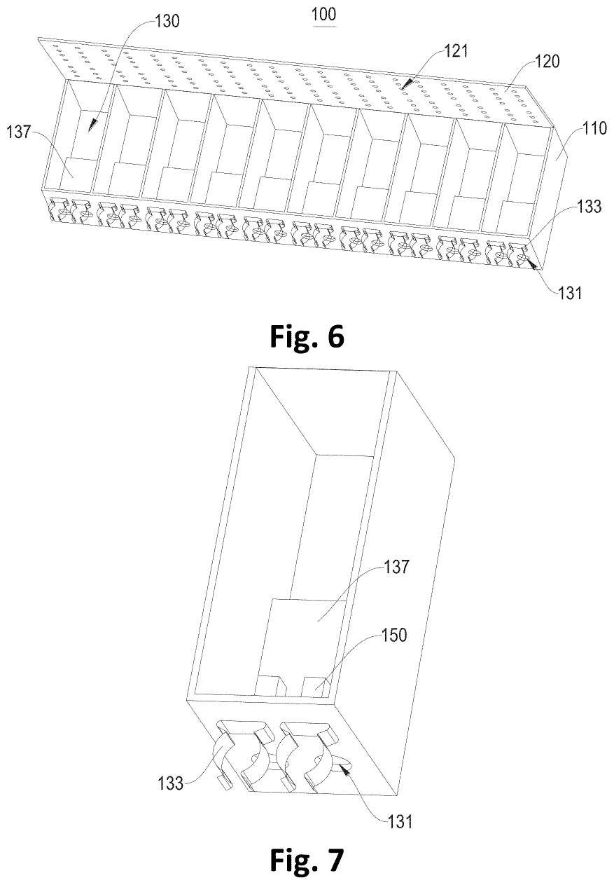

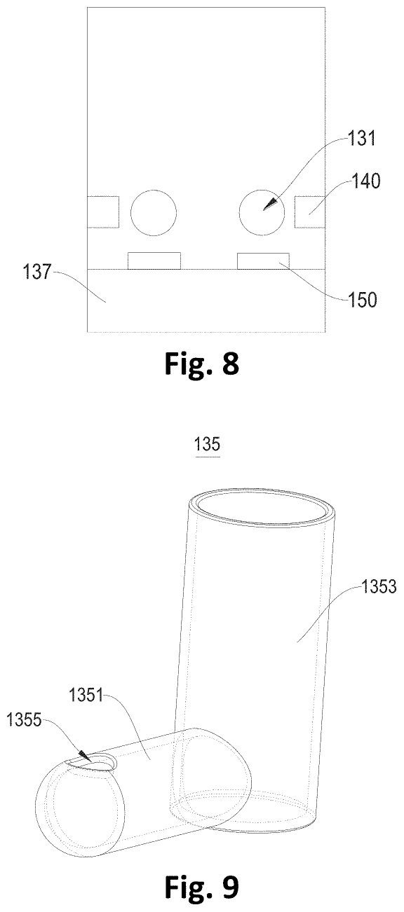

Please refer to FIG. 6, FIG. 7, FIG. 8 and FIG. 9, this embodiment provides a kind of detection device for behavior of animal 100, which consists of a box body 110 and a box cover 120, wherein, the box cover 120 is arranged on the box body 110 and is flexibly connected to the box body. The box body 110 is divided into several chambers 130 by insert plates, which are used to accommodate animals and allow them to move around, and then conduct behavior detection on the animals. In this embodiment, the detection device for behavior of animal 100 is used to detect mice, while in other embodiments of the present invention, the same detection device can be used to detect other experimental animals such as rabbits, and the present invention has not made any restriction on it.

In this embodiment, the box body 110 adopts a rectangular structure and is divided into ten chambers 130 of the same size by nine insert plates. The size of each chamber is 250*105*140 mm. The side walls of the ten chambers 130 on the same side are equipped with two fixing holes 131 respectively, and a fixing frame 133 is arranged above each fixing hole 131. That is, the side wall of the box body 110 is equipped with twenty fixing holes 131 and fixing frames 133. In other embodiments of the present invention, the number of insert plates can be three or seven, and the size of each chamber 130 can be adjusted as required, and the number and the position of the fixing holes 131 and the fixing frame 133 can be adjusted as needed, and the present invention has not made any restriction on it. Such structure can be used to test multiple mice at the same time, and thus saves test time and space, and is convenient for laboratory personnel to operate.

(1) Preparation of 3-hydroxy-3′,4′,5′,5,7-pentamethoxy myricetin (intermediate a):

In this embodiment, the box body 110 is made of acrylic, and both of acrylic and the insert plate are made of opaque material, which may eliminate interference among mice, and has good robustness and low cost, and can be made into different sizes. In addition, the fixing frame 133 is made of stainless steel, and thus can guarantee the stability.

Each chamber 130 is equipped with two feeding bottles 135, which are filled with water and sugar water respectively. Normal mice prefer to drink sugar water, while mice with depression behavior drink water. The depressive behavior of mice will be detected through experimental detection of drinking behavior of mice. The number of feeding bottles 135 matches the number of fixing holes 131 and fixing frames 133. One end of the feeding bottle 135 is connected to the fixing frame 133, and the other end is arranged in the cavity 130 through the fixing hole 131. Such structure can guarantee the stability of the feeding bottle 135, which prevents the mice from touching the feeding bottles 135 and causes water leak, and thus, reduces experimental errors. It should be noted that, in order to ensure the consistency of the feeding bottles 135, the height of the fixing hole 131 in each chamber 130 shall be equal.

Specifically, the feeding bottle 135 adopts an L-shaped structure. The feeding bottle 135 consists of a feeding tube 1351 and a liquid storage tube 1353, wherein, the feeding tube 1351 and the liquid storage tube 1353 are connected at their ends, and the feeding tube 1351 and the liquid storage tube 1353 are vertically connected to each other. The feeding tube 1351 passes through the fixing hole 131 and then is arranged in the chamber 130, and the liquid storage tube 1353 is clamped with the fixing frame 133. The water nozzle 1355 is arranged at one end of the feeding tube 1351 far away from the liquid storage tube 1353, and the opening faces upward. Such structure has a strong leak-proofness, which solves the problem of water leak to a great extent, and makes the experimental results more real and reliable, and thus reduces experimental errors. In this embodiment, the material of the feeding bottle 135 is glass, and preferably, it may be organic glass. In other embodiments of the present invention, the feeding bottle 135 can be made of plastic.

Preferably, the top of the liquid storage tube 1353 is equipped with an opening (not shown in the figure) and a cover matching the opening (not shown in the figure), which allows the drinking water to be added at any time, and is convenient for the laboratory personnel, and thus saves test time.

For the purpose of guaranteeing the accuracy of the experiment, and making sure that the mice drink water consciously. Each chamber 130 is equipped with steps 137, and the steps 137 are arranged on one side close to the fixing hole 131. When the mice drink water, they need to climb the steps 137, and thus avoid drinking action caused by voluntary activities of mice.

Further, the detection device for behavior of animal 100 consists of a sensor 140 and a blocker 150, and each drinking bottle is matched with a sensor 140 and a blocker 150. The sensor 140 is arranged in the chamber 130 and close to the water nozzle 1355 of the feeding bottle 135 to sense the fluctuation of the liquid level at the water nozzle 1355. In this embodiment, the distance between the sensors 140 is 300 mm, and the diameter of the water nozzle 1355 is 22 mm. The blocker 150 is arranged on the steps 137 and close to the feeding bottle 135, which is used to detect the drinking behavior of mice. When the mice approach to the water nozzle 1355 and close to the water surface, the blocker 150 will detect the residence time of the mice. When the mice draw water, the liquid level will fluctuate, and the sensor 140 will sense the specific time and number of drinking water. If the mice stay for more than two seconds, it will be identified as a valid determination, and the drinking time is recorded, and the number of drinking times is increased by 1. Such structure can accurately detect the type of water, the number of drinking times and the drinking time of mice, and thus accurately learn the behavior of mice.

The sensor 140 and the blocker 150 used in the present invention are general equipment in the present technical field, and the present invention has not made any restriction on it.

The box cover 120 is flexibly connected with the box body 110, and the box cover 120 is covered during the experiment to limit the movement range of mice. Wherein, the box cover 120 is equipped with several vents 121, and thus guarantees the air supply of the box body 110 in the process of the test.

The embodiment of the present invention also provides a kind of detection method for behavior of animal, which uses the detection device for behavior of animal 100 to complete detection. The animals used in the embodiments of the present invention may be rabbits, mice and other animals which can be used in experiments. Specifically, it consists of three stages:

The first stage refers to the adaptation stage. Place a bottle of ordinary drinking water and a bottle of 1% sucrose aqueous solution in the breeding cage of the tested animals respectively, and supply food at the same time, and then set aside 46-50 hours to the tested animal for adaptation. After that, place the tested animals in the detection device for behavior of animal 100 to adapt to the new environment.

The second stage refers to the measurement of the baseline. Under the circumstance of not depriving of water and food, each tested animal is given a bottle of ordinary drinking water and a bottle of 1% sucrose aqueous solution respectively, and then conducts baseline measurement lasting for 10-14 hours.

The third stage refers to sucrose preference experiment. After the base value is measured, the tested animals are deprived of water and food for 22-26 hours, and each tested animal is given a bottle of ordinary drinking water and a bottle of 1% sucrose aqueous solution respectively, and then conducts measurement lasting for 14-18 hours.

The measurement process is: each bottle of ordinary drinking water and 1% sucrose aqueous solution are weighed and recorded before and after the test. After the experiment is completed, put the tested animals back to the original cages and give water and food to them. In addition, according to the biological rhythm of the tested animals, all experimental tests will be carried out at night. The embodiment of the present invention takes 1% sucrose water as the test liquid, and the test liquid can be replaced according to the needs of the experiment, and the present invention has not made any restriction on it.

Such method makes the tested animals in the same environment, but separated from each other, and thus eliminates the interference among mice, as well as makes the test environment more stable. It allows to test multiple animals at the same time, and uses a liquid diet feeding bottle with opening at the bottom to simulate the normal feeding and drinking system of mice. Such feeding bottle has strong leak-proofness and solves the problem of water leak to a greater extent, and thus makes the result more real and reliable. Due to the differences between laboratory and laboratory, we have also formulated a corresponding test schedule to ensure the consistency of the experiment as much as possible. In many studies, the process of habituation and the measurement of baseline are often ignored, but these factors should also be taken into account and tested to ensure that the optimal conditions for the sucrose preference experiment are established. At the same time, due to the circadian rhythm will be affected by the drinking behavior of the tested mice, the choose of test time of the sucrose preference experiment is also of great importance, and all stages shall be carried out at night.

Embodiment 2

The present embodiment provides a kind of detection method for behavior of animal, which consists of:

The first stage, which refers to the adaptation stage. Place a bottle of ordinary drinking water and a bottle of 1% sucrose aqueous solution in the breeding cage of mice respectively, and supply food at the same time, and then set aside 46-50 hours to the mice for adaptation. After that, place the mice in the detection device for behavior of animal 100 to adapt to the new environment.

The second stage, which refers to the measurement of the baseline. Under the circumstance of not depriving of water and food, each mouse is given a bottle of ordinary drinking water and a bottle of 1% sucrose aqueous solution respectively, and then conducts baseline measurement lasting for 12 hours.

The third stage, which refers to sucrose preference experiment. After the base value is measured, the mice are deprived of water and food for 24 hours, and each tested animal is given a bottle of ordinary drinking water and a bottle of 1% sucrose aqueous solution respectively, and then conducts measurement lasting for 16 hours.

The measurement process is: each bottle of ordinary drinking water and 1% sucrose aqueous solution are weighed and recorded before and after the test. After the experiment is completed, put the mice back to the original cages and give water and food to them. In addition, according to the biological rhythm of mice, all experimental tests will be carried out at night.

Experimental Example 1 The Detection of Depressive Behavior of Mice

A model of male C57BL/6Ntac mice is modeled by using a classic depression model which is currently widely used, i.e., the chronic unpredictable mild stress model (CUMS), and then uses the detection device for behavior of animal 100 to detect their depressive behavior.

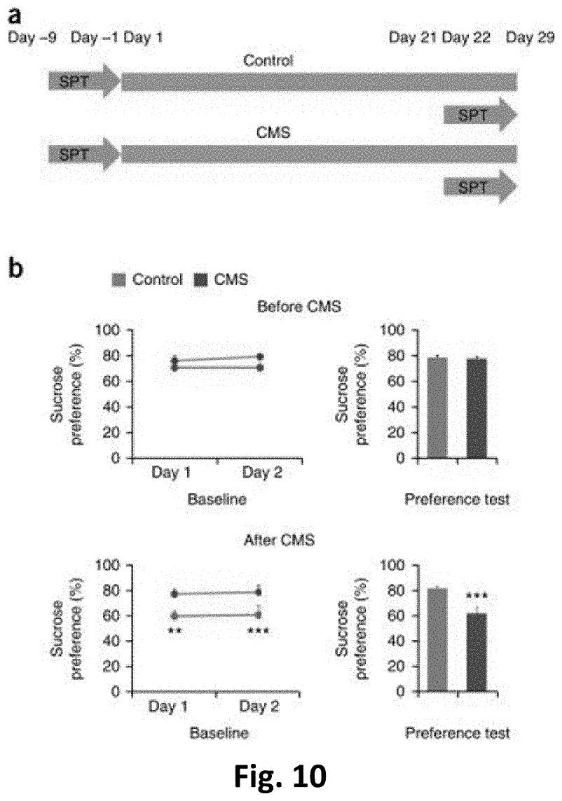

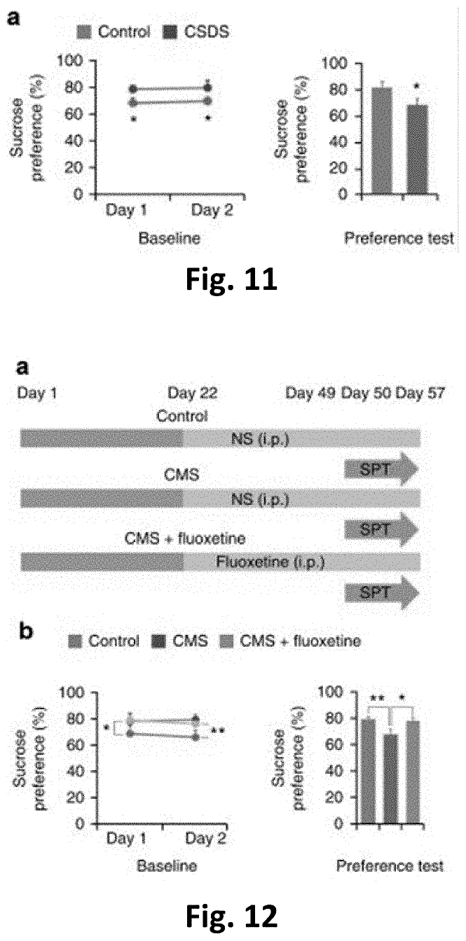

The experimental animals are divided into two groups, namely: the control group and the CUMS group. Eight days prior to the modeling, the base value measurement of the sucrose preference experiment to the two groups of mice was conducted by means of the method provided in the Embodiment 2, and then the CUMS modeling was implemented for 28 consecutive days, in addition, the determination of the sucrose preference experiment was made to the two groups from the 22nd day to the 29th day after modeling. The sucrose preference rate of the mice in the control group reached up to 70-80% (see FIG. 10a). Compared with the control group, the sucrose preference rate of the depressed mice in the CUMS group was significantly reduced to 60% (P<0.001) (see FIG. 5b). Such behavioral results showed that the sucrose preference rate of mice was significantly reduced after CUMS modeling, and thus resulted in a certain degree of anhedonia. Such detection device for behavior of animal can reflect the depressive behavior of mice perfectly. Meanwhile, it is also applicable in another depression model—chronic social defeat stress model (CSDS). From the 11th day to the 18th day after CSDS modelling, the detection of the sucrose preference experiment was conducted on the control group and the CSDS group for a week. The detection results (see FIG. 11a) showed that compared with the normal group, the sucrose preference rate of the mice after CSDS modeling showed a continuous decrease. The above results (see FIG. 11b) showed that the detection device for behavior of animal is suitable for detecting the anhedonia phenomenon produced by various models of depression animals, and can be used as a more sensitive behavioral detection device for the research and development of antidepressant drugs.

Experimental Example 2 The Detection of Anti-Depression Behavior of Mice

In this experiment, male ICR mice were used for CUMS modeling lasting for 56 consecutive days. The experimental animals were divided into three groups, namely: the control group, the CUMS group, and the fluoxetine (intraperitoneal injection, 10 mg/kg)+CUMS group. The drug administration was continued for 24 days from the 22nd day to the 56th day in the process of modeling, and the control mice were performed intraperitoneal injection of normal saline at the same time. After that, from the 50th day to the 57th day in the process of modeling, the detection of the sucrose preference experiment was conducted on the three groups of experimental groups for a week by means of the method provided in the Embodiment 2. The behavioral detection results (see FIG. 12a) showed that compared with the control group, the sucrose preference rate of the mice in model was significantly reduced. When the fluoxetine was given for treatment, the sucrose preference rate of the mice could be increased and returned to a normal level, and thus played certain antidepressant effect. Thus, it can be seen that such detection device for behavior of animal can detect the antidepressant effect by giving drug treatment to the experimental animals.

Experimental Example 3 The Detection of Morphine-Dependent Behavior of Mice

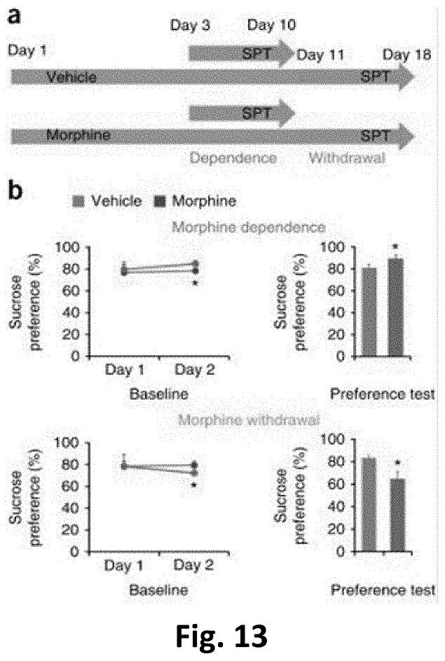

The experimental animals were divided into two groups, namely: the control group and the morphine group. The mice received subcutaneous injections of morphine (10 mg/kg) for 10 consecutive days, and from the 3rd day to the 10th day of administration, the detection of the sucrose preference experiment was conducted on the mice by means of the method provided in the Embodiment 2. The results showed that the sucrose preference rate of the mice in the morphine group was higher than that in the control group, and the method of using the detection device for behavior of animal can identify the behaviors with the nature of rewards, namely: the hyperphagia occurs after abuse of addictive drugs, and the results are shown in the FIG. 13a.

Experimental Example 4 The Detection of Morphine Withdrawal Behavior of Mice

The experimental animals were divided into two groups, namely: the control group and the morphine group. The mice received subcutaneous injections of morphine (10 mg/kg) for 10 consecutive days, and from the 11th day to the 17th day, the morphine injection was stopped, and the detection of the sucrose preference experiment was conducted on the mice by means of the method provided in the Embodiment 2 at the same time. The behavioral detection showed that when the mice were not given morphine, the sucrose preference rate of them was lower than that of normal control mice, and the detection device for behavior of animal is able to detect the withdrawal behavior of addicted mice after drug withdrawal, and the results are shown in the FIG. 13b.

Although the present invention has been described in detail hereinbefore with general descriptions and specific embodiments, some modifications or improvements can be made based upon the present invention, which is obvious to those skilled in the art. Therefore, these modifications or improvements made without departing from the spirit of the present invention shall fall within the protection scope of the present invention.

1. A kind of monitoring method for drinking behavior of laboratory mice, characterized in that consisting of following steps:
Data collection: to obtain the raw data of drinking of mice, and store the said raw data;
Data filtering: to conduct data filtering on the said raw data to filter out false data of jitter from the said raw data, wherein, the said false data of jitter consists of the data triggered by measurement signal transient and jitter caused by accidentally touch of the mice body, and their exploratory action;
Data statistics: to complete index statistics and analysis of the raw data filtered out the said false data of jitter, and thus form indicator data, as well as store the said indicator data;
Data presentation: the said indicator data is displayed in classification of real-time data, historical data, and report data.

2. The said kind of monitoring method for drinking behavior of laboratory mice according to claim 1, characterized in that during the said data collection process, setting communication parameter and identifying communication port according to the selected operation object, and then receiving data collection instructions; after that, according to the current status of the said operation object and the mutual exclusion status among the objects to determine the validity of the instruction, and then, integrating effective instructions into a data packet of instructions for execution.

3. The said kind of monitoring method for drinking behavior of laboratory mice according to claim 2, characterized in that the status data and sampling data are fed back during the executive process of the said data packet of instructions, and then, inputting the said status data and sampling data in the data buffer pool, and detecting the integrity and correctness of the said data packet of instructions, as well as analyzing the valid data points of the said instruction data for utilization.

4. The said kind of monitoring method for drinking behavior of laboratory mice according to claim 1, characterized in that during the said data filtering process, filtering the data triggered by measurement signal transient and jitter caused by accidentally touch of the mice body, and their exploratory action through non-false judgment method, and the statistical model of real drinking behavior of mice is used to match the real drinking data of mice.

5. The said kind of monitoring method for drinking behavior of laboratory mice according to claim 1, characterized in that during the said data presentation process, the data presentation methods include in the forms of animation, numerical value, list, curve graph, pie chart, image, and document.

6. A kind of monitoring system for drinking behavior of laboratory mice, characterized in that consisting of
A data collection module, which is used to obtain the raw data of drinking of mice, and store the said raw data;
A data filtering module, which is used to conduct data filtering on the said raw data to filter out false data of jitter from the said raw data, wherein, the said false data of jitter consists of the data triggered by measurement signal transient and jitter caused by accidentally touch of the mice body, and their exploratory action;
A data statistics module, which is used to complete index statistics and analysis of the raw data filtered out the said false data of jitter, and thus form indicator data, as well as store the said indicator data;
A data presentation module, which is used to display the said indicator data in classification of real-time data, historical data, and report data.

7. The said kind of monitoring system for drinking behavior of laboratory mice according to claim 6, characterized in that the said data collection module is equipped with a data transceiver unit and a validity judgment unit, and the said data transceiver unit is used to set communication parameter and identify communication port according to the selected operation object, and then receive data collection instructions; after that, the said validity judgment unit is used to determine the validity of the instruction according to the current status of the said operation object and the mutual exclusion status among the objects, and then, integrate effective instructions into a data packet of instructions for execution.

8. The said kind of monitoring system for drinking behavior of laboratory mice according to claim 7, characterized in that the said data collection module is further equipped with a data buffer unit, a data detection unit and a data analysis unit, wherein, the said data buffer unit is used to feed back the status data and sampling data during the executive process of the said data packet of instructions, and then, input the said status data and sampling data in the data buffer pool; and the said data detection unit is used to detect the integrity and correctness of the said data packet of instructions, as well as the said data analysis unit is used to analyze the valid data points of the said instruction data for utilization.

9. The said kind of monitoring system for drinking behavior of laboratory mice according to claim 6, characterized in that the said data filtering module consists of a false data filtering unit and a data matching unit, wherein, the said false data filtering unit is used to filter the data triggered by measurement signal transient and jitter caused by accidentally touch of the mice body, and their exploratory action through non-false judgment method;
and the said data matching unit is used to match the real drinking data of mice by using the statistical model of real drinking behavior of mice.

10. A kind of detection device for drinking behavior of laboratory mice, characterized in that consisting of:
A box body, and the said box body is divided into at least two chambers by at least one insert plate, and the said chambers are used to accommodate animals and allow the said animals to move around, furthermore, each of the said chambers is equipped with at least two fixing holes on the side walls, and a fixing frame is arranged above each said fixing hole;
Each of the said chambers is equipped with steps, and the said steps are arranged on one side close to the said fixing hole;
Four feeding bottles at least, wherein, one end of each of the said feeding bottle is connected to one of the said fixing frame, and the other end is set in the said chamber through the said fixing hole under the said fixing frame;
Four sensors at least, with function of data collection, and each of the said feeding bottles is equipped with the said sensor, and the said sensor is arranged in the said chamber close to the water nozzle of the said feeding bottle, which is used to sense the fluctuation of the liquid level at the said water nozzle, and record the number of times and the duration of the fluctuation of the water level;
A box cover, and the said box cover is arranged on the said box body and is flexibly connected to the box body, and the said detection device for behavior of animal further consists of four blockers at least, and each of the said feeding bottle is equipped with one said blocker, and the said blocker is arranged on the said steps and close to the said feeding bottle.
